(19) United States
(12) Patent Application Publication
(10) Pub. No.: US 2025/0325959 A1
(43) Pub. Date: Oct. 23, 2025

(54) MATERIAL FOR TRAPPING SUBSTANCE IN GAS PHASE, AND METHOD FOR TRAPPING SUBSTANCE IN GAS PHASE

(71) Applicant: National University Corporation Tokyo Medical and Dental University, Tokyo (JP)

(72) Inventors: Naoya Takeda, Tokyo (JP); Misa Nakaya, Tokyo (JP); Tsubomi Tomono, Tokyo (JP); Yuji Tsuchido, Tokyo (JP); Kohji Mitsubayashi, Tokyo (JP); Kenta Iitani, Tokyo (JP); Takahiro Arakawa, Tokyo (JP); Koji Toma, Tokyo (JP)

(73) Assignee: National University Corporation Tokyo Medical and Dental University, Tokyo (JP)

(21) Appl. No.: 18/580,755

(22) PCT Filed: Jul. 26, 2022
(86) PCT No.: PCT/JP2022/028692

§ 371 Date: Mar. 3, 2025

(30) Foreign Application Priority Data
Jul. 27, 2021 (JP) 2021-122143

(51) Int. Cl. B01J 20/26; B01D 53/04; G01N 33/98

(52) U.S. Cl. CPC B01J 20/265; B01D 53/04; G01N 33/98; B01D 2253/202; B01D 2257/708

(57) ABSTRACT

The purpose of the invention is to provide a gasborne substance trapping material for trapping gasborne a substance, preferably a volatile organic compound (VOC), which is greatly simplified with respect to the production and application processes, requires no extemporaneous compounding, can be produced within a short time, and can be stored for a long period of time. Specifically, provided is a gasborne substance trapping material for trapping a substance contained in a gas phase, wherein the gasborne substance trapping material contains a water-soluble polymer having, included therein, a functional protein that retains a capability to bind to the substance, wherein the functional protein exhibits a capability to bind to the substance to thereby trap the substance.

### TECHNICAL FIELD

The present invention relates to a gasborne substance trapping material for trapping a target substance contained in a gas phase, wherein the material is composed of a polymeric material into which a functional protein is mixed, and a gasborne substance trapping method for trapping a target substance contained in a gas phase using the gasborne substance trapping material.

### BACKGROUND ART

Living organisms discharge various types of volatile organic compounds (VOCs) as biological gases in association with biological activities. The biological gas encompasses a gas emitted from, for example, a plant in a broad sense. However, in a narrow sense, this term refers to a gas discharged from human. This biological gas as used herein in the narrow sense can roughly be categorized into gases originating from skin, exhaled breath, intestine, blood and urine.

Of these biological gases, exhaled gases are often utilized clinically. The reasons are that an exhaled gas contains various types of components of 200 to 300 species, is useful as biological information that is distinctive from that of blood components, allows non-invasive sampling, and allows a patient having no medical knowledge to readily measure by himself/herself. Examples of breathalyzers in practical use include those that measure EtOH (ethanol) for checking sobriety, ¹³CO₂ (stable isotope) as a tester for H. pylori infection of the stomach, NO (nitric oxide) as a therapeutic monitor for asthma, H₂ (hydrogen) as a monitor for lactose intolerance, CO (carbon monoxide) as a smoking monitor.

In view of these circumstances, a device for measuring EtOH in an exhaled breath has been developed (Patent document 1). This device employs a gasborne substance trapping material in which an alcohol dehydrogenase (ADH) is immobilized via physical adsorption to a cotton mesh to which Nicotinamide adenine dinucleotide (NAD⁺) as a coenzyme is added for the sake of visualization. When the cotton mesh is exposed to ethanol (EtOH) gas contained in, for example, the skin gas, enzymic activity reactions of the ADH breaks EtOH, as being a substrate, into acetaldehyde, which in turn reduces NAD⁺ to thereby convert it into the NADH. This NADH is excited by the ultraviolet light of 340 nm wavelength to radiate a fluorescence of 490 nm wavelength by which the EtOH gas is measured via monitoring this fluorescence.

Unfortunately, this device employing a cotton mesh for measuring EtOH in an exhaled breath has defects including: 1) It necessitates an enzyme immobilization process having a multiple of processes in few hours scale to manufacture the device, and therefore a large-scale production is difficult; and 2) A user needs to drop a coenzyme solution into the enzyme membrane just before use, that is, it requires extemporaneous compounding. Accordingly, desired is the development of a gasborne substance trapping material, which is greatly simplified with respect to the production and application processes, requires no extemporaneous compounding, can be produced within a short time, and can be stored for a long period of time.

### PRIOR ART DOCUMENTS

### Patent Documents

[Patent document 1] WO2019/103130A1

### SUMMARY OF THE INVENTION

### Problems to be Solved by the Invention

The object of the invention is to provide a gasborne substance trapping material for trapping gasborne target substances, particularly volatile organic compounds (VOCs), which is greatly simplified with respect to the production and application processes, can be produced within a short time, requires no extemporaneous compounding, and can be stored for a long period of time.

### Means to Solve the Problems

The inventors have found out as a result of their diligent research that, when a solution prepared by mixing a water-soluble polymeric material and functional molecules such as proteins (e.g., enzymes) is solidified and processed in a suitable manner so as to allow the functional molecules to be incorporated in the water-soluble polymeric material, there can be expressed a function inherent in the functional molecule that does not normally exhibit activity in a gas phase, and therefore have developed a gasborne substance trapping material as a composite material in which the functional molecules are incorporated in the water-soluble polymeric material.

More specifically, provided is a gasborne substance trapping material, which can trap a substance contained in a gas phase, the gasborne substance trapping material comprising a water-soluble polymer having, included therein, a functional protein that retains a capability to bind to the substance, wherein the functional protein exhibits a capability to bind to the substance to thereby trap the substance.

In the gasborne substance trapping material according to the present invention, the functional protein in the water-soluble polymer may retain its functionality.

In the gasborne substance trapping material according to the present invention, the functional protein may be an enzyme or an antibody.

In the gasborne substance trapping material according to the present invention, the substance may be a volatile organic compound (VOC).

In the gasborne substance trapping material according to the present invention, the functional protein may be an enzyme, and the water-soluble polymer may further be mixed with a cofactor.

In the gasborne substance trapping material according to the present invention, the water-soluble polymer may be selected from polyvinyl alcohol (PVA), polyethylene oxide (PEO), polyacrylamide (PAAm), polyvinylpyrrolidone (PVP), and dextran and polyethylene glycol (PEG).

In the gasborne substance trapping material according to the present invention, the water-soluble polymer may be in the form of a microfiber or a thin film.

In the gasborne substance trapping material according to the present invention, the volatile organic compound may be ethanol, the enzyme may be an alcohol dehydrogenase, and the cofactor may be the NAD⁺ (Oxidized Nicotinamide adenine dinucleotide).

The present invention also provides a biosensor for trapping and measuring a substance contained in a gas phase, wherein the biosensor employs the gasborne substance trapping material.

In the biosensor according to the present invention, the substance may be a volatile organic compound.

The present invention also provides a method for trapping a substance contained in a gas phase with a water-soluble polymer having a functional protein, wherein the functional protein exhibits a capability to bind to the substance to thereby trap the substance.

In the method according to the present invention, the substance may be a volatile organic compound.

### Effects of the Invention

The present invention may provide a gasborne substance trapping material for trapping a substance such as volatile organic compound (VOC) contained in a gas phase, which is greatly simplified with respect to a production process and an application process, can be produced within a short time, does not require extemporaneous compounding, and can be stored for a long period of time.

### BRIEF DESCRIPTION OF THE DRAWINGS

FIG. 1 shows a diagram illustrating a detection mechanism of EtOH gas based on a reaction between EtOH and alcohol dehydrogenase (ADH) in a gasborne substance trapping material that employs the ADH for trapping and detecting EtOH contained in a gas phase.

FIG. 2 shows a diagram illustrating an outline of a method for manufacturing the gasborne substance trapping material for trapping EtOH contained in a gas phase.

FIG. 3 shows a diagram illustrating an experimental system for measuring EtOH gas (upper figure), and settings (lower figure) of a distance between an excitation light source and an excitation target object (or the gasborne substance trapping material), and a distance between a Pasteur pipet (gas ejection opening) and an enzyme membrane (or the gasborne substance trapping material)

FIG. 4 shows a fluorescence image that depicts the fluorescence (Excitation light wavelength: 340 nm; Emission light wavelength: 490 nm) when an air having EtOH was applied to a microfiber mesh of polyvinyl alcohol (PVA) into which ADH and NAD⁺ were mixed.

FIG. 5 shows a graph illustrating variations in fluorescence intensity (Excitation light wavelength: 340 nm; Emission light wavelength: 490 nm) over time when (1) an air containing 200 ppm EtOH was applied to a microfiber mesh (PVA/ADH/NAD⁺ mesh) formed of polyvinyl alcohol (PVA) into which ADH and NAD⁺ were mixed; and (2) when, as a negative control, an air containing no EtOH was applied to the PVA/ADH/NAD⁺ mesh, and (3) when, as a negative control, an EtOH-containing air was applied to a microfiber mesh (PVA/NAD⁺ mesh) formed of polyvinyl alcohol (PVA) into which NAD⁺ was mixed without mixing ADH.

FIG. 6 shows a graph illustrating variations in differential values of the variations in fluorescence intensity over time or the rates of fluorescence variations when airs containing 200 ppm EtOH gases were applied in the respective flow rates to the PVA/ADH/NAD⁺ mesh.

FIG. 7 shows a graph illustrating a correlation between variations in the differential value of the variations in fluorescence intensity over time (the rates of fluorescence variations) and the applied amounts of EtOH per unit time when EtOH-containing airs were applied in the respective flow rates to the PVA/ADH/NAD⁺ mesh.

FIG. 8A is a scanning electron microscope image of the PVA/ADH/NAD⁺ mesh on the day of fabrication in an experiment for studying the preservation stability of the PVA/ADH/NAD⁺ mesh.

FIG. 8B is a scanning electron microscope image of the PVA/ADH/NAD⁺ mesh after preserving it for two weeks at 4° C. after fabrication in an experiment for studying the preservation stability of the PVA/ADH/NAD⁺ mesh.

FIG. 9 is a graph showing responses (variations in fluorescence intensity over time) to the application of EtOH (20 seconds application) in an experiment for studying preservation stability of the PVA/ADH/NAD⁺ mesh on the day of fabrication and after preserving it for two weeks at 4° C. after fabrication.

FIG. 10 shows a graph illustrating variations in fluorescence intensity (Excitation light wavelength: 340 nm; Emission light wavelength: 490 nm) over time when an air containing 200 ppm EtOH was applied for 20 seconds to microfiber meshes of the respective water-soluble polymers into which ADH and NAD⁺ were mixed. PVA represents polyvinyl alcohol, PAAm represents polyacrylamide, PEO represents polyethylene oxide, and PVP represents polyvinylpyrrolidone.

### MODE FOR CARRYING OUT THE INVENTION

### 1. Gasborne Substance Trapping Material, and Biosensor Employing the Gasborne Substance Trapping Material

### One Aspect of the Present Invention is a Gasborne Substance Trapping Material.

The gasborne substance trapping material serves as a composite material which can be made by solidifying and processing a solution that is prepared by mixing a water-soluble polymeric material with a functional molecule such as protein (e.g., an enzyme) by a suitable method to thereby allow the functional molecule to be incorporated in the water-soluble polymeric material, and the material allows the functional molecule to exhibit a function inherent in the functional molecule that does not normally exhibit activity in a gas phase.

Functional molecules such as enzymes or antibodies normally express their functions in a liquid phase. The term “gasborne substance trapping material” as used herein refers to a composite material in which a functional molecule is incorporated in a water-soluble polymeric material, where the material can make a target substance, contained in a gas phase, bind to the functional molecule for trapping the substance based on a function inherent in the functional molecule in a solid phase rather than in a liquid phase, the target substance being substances such as viruses or organic compound molecules including volatile organic compound molecules. In the case when the functional molecule is, for example, an enzyme, the term “gasborne substance trapping material” according to the present invention also encompasses not only a material to make a target material bind to the enzyme for trapping the substance but also a material to express a catalyst activity of the enzyme.

Examples of the gas phase include, but are not particularly limited to, normal air that contains a target molecule as well as a biological gas such as an exhaled breath or skin gas discharged from a living organism.

The wording “retains a function” as used herein is used in a qualitative sense, meaning that it has the same quality of function before and after the comparison. In other words, the wording means not only that the same quality of function is maintained to the same or higher level, but also that the function is retained without being deactivated, as long as the same quality of function can be performed, even if the function is quantitatively reduced.

The incorporation of functional molecules such as proteins into a polymer material generally eliminates or significantly reduces the functionality that is innate to the functional molecule. The inventors have reached a finding that a water-soluble polymer may be used as a polymer material to retain the functionality of functional molecules such as proteins, and have found out that a water-soluble polymer material with which the functional molecules were mixed can be used to trap a target material contained in a gas phase in a favorable manner. The present invention is based on this finding.

The gasborne substance trapping material is more specifically directed to a gasborne substance trapping material for trapping a substance contained in a gas phase, comprising a water-soluble polymer having a functional protein that retains a capability to bind to the substance, wherein the functional protein exhibits a capability to bind to the substance to thereby trap the substance.

Moreover, this gasborne substance trapping material may be used as a gas sensor, particularly a biosensor, to trap a target substance contained in a gas phase for analyzing the same but the material is not limited to such material.

The term “biosensor” as used herein refers to a sensor for analyzing or detecting a detection target substance using molecular discrimination functionality of a biologically relevant substance such as an enzyme, microorganism or antibody.

According to the gasborne substance trapping material of the present invention, examples of the target substance to be trapped include volatile organic compounds (VOCs). For example, the VOCs, contained in a biological gas such as an exhaled breath or skin gas discharged from a living organism, may be trapped using the gasborne substance trapping material of the present invention and the material may be used as a biosensor for measuring an amount of the trapped VOCs, which can therefore be used to diagnose, for example, physiological conditions and/or diseases of the living organism.

For example, an exhaled breath contains 70 to 2000 ppb of EtOH, 3 to 90 ppb of acetaldehyde, 100 to 2300 ppb of methanol, 200 to 900 ppb of acetone, 50 to 250 ppb of isopropanol, 48 to 83 ppb of formaldehyde, and 400 to 1350 ppb of ammonia, and less than 50 ppb of dimethyl sulfide.

Accordingly, examples of the biosensor using the gasborne substance trapping material of the present invention include, but are not particularly limited to, a biosensor for measuring ethanol (EtOH) for alcoholism treatment, a biosensor for measuring acetaldehyde for risk assessment of oral or esophageal cancer, a biosensor for measuring methanol for evaluation of intestinal environment, a biosensor for measuring acetone for evaluation of diabetes and lipid metabolism, a biosensor for measuring isopropanol for evaluation of diabetes and lipid metabolism, a biosensor for measuring formaldehyde for diagnosis of lung cancer, a biosensor for measuring ammonia for diagnosis of liver diseases and a biosensor for measuring dimethyl sulfide for evaluating halitosis or oral environment.

Further, the gasborne substance trapping material of the present invention may be used not only as a gas sensor or a biosensor but also for cleaning the air by trapping a substance that is floating in the air. Further, the gasborne substance trapping material of the present invention may be used for recovering a desired substance by selectively trapping such substance that is floating in the air. Moreover, the cleanliness and contamination levels of the gas in the air can be measured by trapping and/or recovering the floating substance in the air for analysis. Furthermore, the trapping of a pathogen substance floating in the air may prevent affection with a disease

In the gasborne substance trapping material according to the present invention, preferable examples of the functional protein include an enzyme and an antibody among which an enzyme is more preferred.

In the gasborne substance trapping material according to the present invention, when the functional protein is, for example, an enzyme, the material may be prepared by further mixing a cofactor to the polymer. As an example of the cofactor, a coenzyme is preferred.

In the gasborne substance trapping material according to the present invention, as an example of the water-soluble polymer, the water-soluble polymer may be selected from, but is not limited to, polyvinyl alcohol (PVA), polyethylene oxide (PEO), polyacrylamide (PAAm), polyvinylpyrrolidone (PVP), dextran and polyethylene glycol (PEG).

In the gasborne substance trapping material according to the present invention, the water-soluble polymer may preferably be in the form of a microfiber or thin film for the purpose of widening the contact area on which the VOCs are in contact when a gas such as exhaled breath or skin gas, containing the VOCs as measurement targets, is applied to the gasborne substance trapping material. A microfiber or thin film that enables the widening of the contact area may be used to precisely and accurately detect the VOCs as measurement targets. As an example, when the gasborne substance trapping material is used in the form of a microfiber, the material may be used as a microfiber mesh to ensure favorable permeability of the VOCs-containing gas to thereby trap a small amount of VOCs, contained in the gas, into the gasborne substance trapping material.

The gasborne substance trapping material according to the present invention may be manufactured as a microfiber mesh by using a mixture solution of a polymer material into which a functional polymer is mixed or using a water-soluble polymer mixture into which not only the functional polymer but also a cofactor is mixed, and performing spinning using any suitable technique. The preferable technique for performing the spinning includes, but is not particularly limited to, electrospinning.

The electrospinning allows the manufacturing of a three-dimensional structure composed of microfibers containing both or either one of a protein and a cofactor, and this structure also serves as a gasborne substance trapping material. The three-dimensional structure has the following advantages:
1) The capturing rate increases in association with an increment in the space occupation ratio of the gasborne substance trapping material.
2) The three-dimensional structure allows a one-time measurement of three-dimensional data while a sensor of sheet-shaped trapping material limits a distribution measurement of the gasborne components to that of two-dimensional data. Here, a three-dimensional structure of microfiber may be used to fabricate a three-dimensional structure having gas permeability suitable for measuring components contained in a gas phase.

Example applications of the gasborne substance trapping material of the present invention include a biosensor that targets ethanol which is a volatile organic compound (VOC), where the biosensor is of a water-soluble polymer material into which an enzyme of alcohol dehydrogenase (ADH) and a cofactor of NAD⁺ (Oxidized Nicotinamide adenine dinucleotide) are mixed.

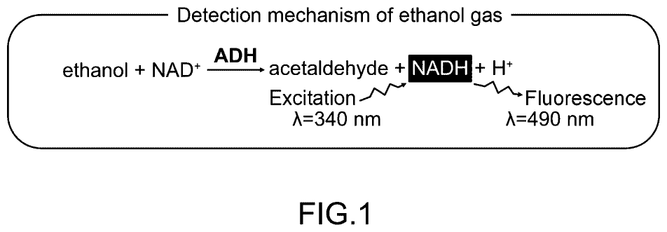

According to this biosensor that uses a gasborne substance trapping material for detecting EtOH, which is formed of a water-soluble polymer into which the ADH and NAD⁺ are mixed, when EtOH is bonded to the ADH, and EtOH is dehydrogenated into acetaldehyde, the NAD⁺ emits fluorescence of the NADH (Excitation light wavelength: 340 nm; Emission light wavelength: 490 nm) which can therefore be measured for the detection of EtOH with a high degree of sensitivity (See FIG. 1).

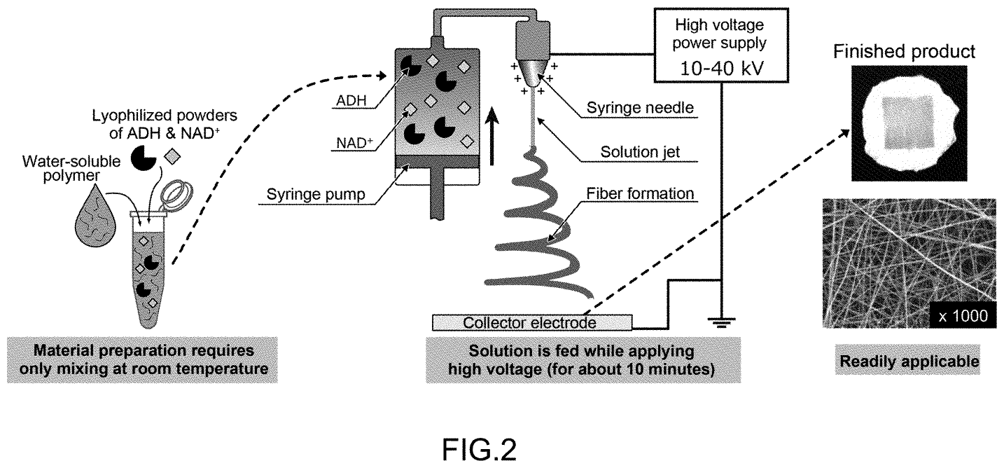

The gasborne substance trapping material for use in this EtOH-detecting biosensor may be manufactured as a microfiber mesh by using, for example, an aqueous solution of water-soluble polymer material into which lyophilized powders of ADH and NAD⁺ are mixed such that the solution is fed while applying a high voltage by electrospinning to form a microfiber on a collector electrode (See FIG. 2).

An EtOH-containing gas may be applied to the microfiber mesh formed of the water-soluble polymer into which ADH and NAD⁺ are incorporated, and the produced fluorescence of the NADH may be measured to utilize the mesh as a biosensor for detecting the gas-borne EtOH.

As a method for analyzing the measured fluorescence, there may be used methods of:
(1) Measuring and analyzing a variation in fluorescence intensity over time (See working examples and FIG. 5), and
(2) Analyzing a rate of fluorescence variation, which is a variation per unit time of the variation in fluorescence intensity over time, i.e., analyzing differential values of the variation in fluorescence intensity over time as set forth in the item (1) shown above.

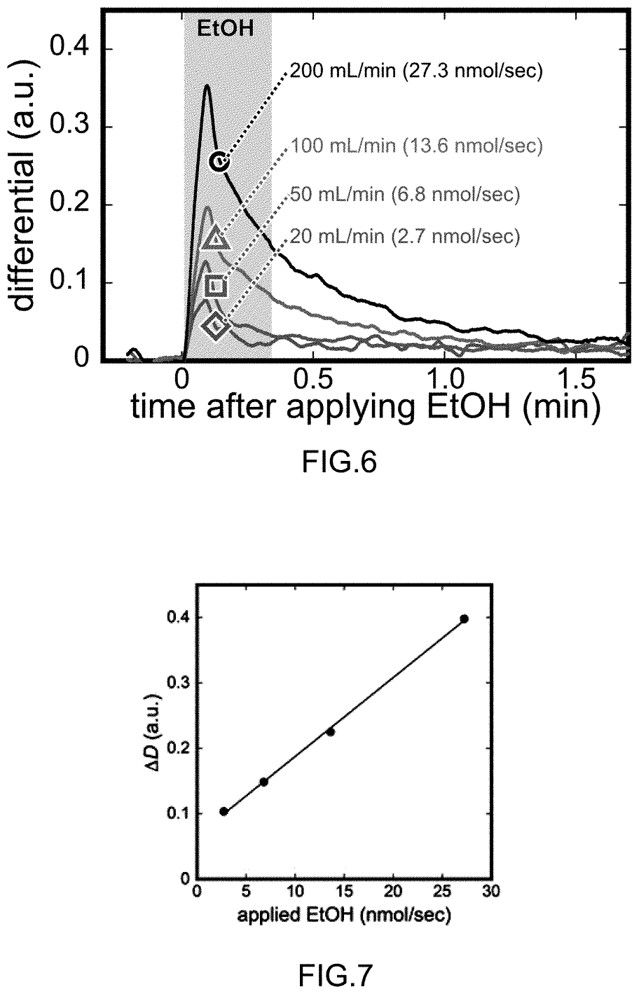

The differential values as set forth in the item (2) of the variation in fluorescence intensity over time indicate production amounts of the NADH per unit time, and this analyzing method can more accurately reflect the amount of the detected EtOH (See working examples and FIG. 6).

This gasborne substance trapping material formed of the water-soluble polymer incorporating the ADH and NAD⁺ which target EtOH as a target substance and the EtOH measuring biosensor using the same can be used as a micromesh immediately after the manufacturing unlike the EtOH detecting biosensor (Patent document 1) that employs a cotton mech which necessitates extemporaneous compounding of the NAD⁺ solution and constant supply during detection. Moreover, as shown in the following working examples, it has excellent preservation stability and requires no extemporaneous compounding.

### 2. Method for Capturing Gasborne Substance

A further aspect of the present invention is directed to a method for capturing a gasborne substance. The invention is more specifically directed to a method for trapping a substance contained in a gas phase with a water-soluble polymer having a functional protein having a function to bind to a target substance, wherein the functional protein exhibits a capability to bind to the substance for trapping the substance contained in a gas phase.

The trapping method according to the present invention is directed to a method that allows a functional molecule, having a capability to bind to a target substance, to be incorporated in a water-soluble polymeric material, and apply a gas, containing the target substance, to the water-soluble polymeric material to make the functional molecule, incorporated in the water-soluble polymeric material, trap the target substance in a gas by keeping the binding capability.

In the trapping method according to the present invention, examples of the gas phase include a biological gas such as an exhaled breath or skin gas discharged from a living organism. Examples of the substance include volatile organic compounds (VOCs), and examples of the functional protein include an enzyme and an antibody.

In the trapping method according to the present invention, examples of the water-soluble polymer include polyvinyl alcohol (PVA), polyethylene oxide (PEO), polyacrylamide (PAAm), polyvinylpyrrolidone (PVP), dextran and polyethylene glycol (PEG).

The trapping method according to the present invention may be utilized for a biosensor for measuring VOCs in a biological gas for the purposes of diagnosing a physiological condition and/or a disease of a living organism. Examples of the biosensor utilizing the trapping method of the present invention include, but are not particularly limited to, a biosensor for measuring ethanol (EtOH) for alcoholism treatment, a biosensor for measuring acetaldehyde for risk assessment of oral or esophageal cancer, a biosensor for measuring methanol for evaluation of intestinal environment, a biosensor for measuring acetone for evaluation of diabetes and lipid metabolism, a biosensor for measuring isopropanol for evaluation of diabetes and lipid metabolism, a biosensor for measuring formaldehyde for diagnosis of lung cancer, a biosensor for measuring ammonia for diagnosis of liver diseases and a biosensor for measuring dimethyl sulfide for evaluating halitosis or oral environment

Examples of the EtOH measurement biosensor that targets ethanol (EtOH) as a VOC include a biosensor that utilizes a gasborne substance trapping material formed by spinning a water-soluble polymer material into which an enzyme of alcohol dehydrogenase (ADH) and a cofactor of NAD⁺ (Oxidized Nicotinamide adenine dinucleotide) are mixed.

As already explained above, the gasborne substance trapping material formed of the water-soluble polymer into which the ADH and NAD⁺ are mixed can measure the amount of EtOH contained in a gas phase by observing a fluorescence of the NADH (Excitation light wavelength: 340 nm; Emission light wavelength: 490 nm) which is emitted from the NAD⁺ when the ADH dehydrogenates EtOH into acetaldehyde.

It is preferred in terms of allowing a target molecule contained in a gas phase to be favorably trapped that gasborne substance trapping material for use in the method of the present invention have a large contact area in contact with the gas, and be in the form of a microfiber or a thin film. It is also preferred in terms of ensuring a favorable gas permeability that the material be in the form of a microfiber mesh.

Further, as already explained above, the biosensor utilizing the method of the present invention requires no extemporaneous compounding of a reagent to be used, and the biosensor may be manufactured in the form of a microfiber for immediate use by using, for example, a water-soluble polymer solution into which the ADH and NAD⁺ are mixed, and spinning it by electrospinning. Further, this biosensor in the form of a microfiber also has a property of excellent preservation stability.

### WORKING EXAMPLES

All documents mentioned herein are incorporated by reference in their entirety. The working examples described herein are illustrative of embodiments of the invention and should not be construed as limiting the scope of the invention.

### Working Example 1

Gasborne EtOH detection by visualization using microfiber mesh in which the (alcohol dehydrogenase) ADH and NAD⁺ are incorporated in polyvinyl alcohol (PVA)

### 1. Materials and Devices

### (1) Experimental Reagents and Instruments

The following experimental reagents, instruments, and devices were used.
Terumo Injection Needle 22G 1 1/2 RB (Terumo Corporation, NN-2238R)
Disposable syringe 1 mL (Terumo Corporation, SS-01T)
Polyvinyl alcohol solution (PVA solution) (YAMATO CO., LTD., Arabic Yamato (Registered Trademark) Liquid Glue)
ADH: Alcohol Dehydrogenase from Saccharomyces cerevisiae (Sigma-Aldrich, Cat No. A7011)
NAD⁺: β-Nicotinamide-adenine dinucleotide oxidized form (Oriental Yeast Co., ltd, Cat No. 44056000)
Sodium hydroxide (FUJIFILM Wako Pure Chemical Corporation, Cat No. 198-13765)
MilliQ water
Ethanol (FUJIFILM Wako Pure Chemical Corporation, Cat No. 056-06967)

### (2) Experimental Devices

Spinning device (MECC CO., LTD., NANON-03)
Magnetron sputtering system (Vacuum device Corp, MSP-10)
Scanning electron microscope (KEYENCE CORPORATION, VE-9800).
Benchtop pH/water quality analyzer (HORIBA Advanced Techno, Co., Ltd., LAQUA, pH/ION METER F-72)
Monochrome CMOS Camera (Thorlabs Inc., CS235MU)

### 2. Fabrication of Microfiber Mesh Incorporating ADH and NAD⁺

(1) 50 mL MilliQ water was put into a beaker and stirred by a stirrer. 2 g of sodium hydroxide was added in small batches to prepare 1 M NaOH.
(2) The 1M NaOH as prepared in (1) was added into a PVA solution (having a pH of about 4) to adjust the solution to the pH of 8. For this purpose, a benchtop pH/water quality analyzer, corrected using three types of pH standard solutions (pH of 4, 7 and 9), was used.
(3) ADH and NAD+ were directly added into the PVA solution as prepared in (2) having a pH of 8 to have ADH concentration of 6.2 mg/mL and NAD⁺ concentration of 13.3 mg/mL. The solution concentrations were adjusted such that the amount of NAD⁺ was set as 1 μmol and the amount of ADH was 100 units when the spinning amount per one sheet was set as 50 μL. Their specific compositions are as shown in Table 1. A polyvinyl alcohol solution having NAD⁺ concentration of 13.3 mg/mL was also prepared in the same way as a negative control.

TABLE 1
Prepared concentrations, spinning amounts
and contents per sheet of ADH and NAD

| | ADH | NAD⁺ |
|---|---|---|
| Concentration (mg/ml) | 6.2 | 13.25 |

| | |
|---|---|
| Flow rate (ml/min) | 0.015 |
| Spinning time (min) | 3.3 |
| Spinning amount (ml) | 0.05 |

| | | |
|---|---|---|
| Content (mg) per sheet | 0.31 | 0.66 |
| ADH activity (Units/mg) | 3.21 | — |
| Enzyme activity per sheet (Units) | 100 | — |
| Molecular mass of NAD⁺ (g/mol) | — | 663.43 |
| Molar number of NAD⁺ per sheet | — | 1 |

(4) The solution as prepared in 3) shown above was used to spin a fiber using electrospinning to thereby fabricate a microfiber mesh. The electrospinning conditions were as shown in Table 2. As a collector, there was used a resin seat having a 1.5 cm by 1.5 cm square hole on which aluminum foil was covered.

TABLE 2
Electrospinning condition

| | |
|---|---|
| Flow rate (ml/min) | 0.015 |
| Spinning time (min) | 3.3 |
| Spinning amount (ml) | 0.05 |
| Spinning height (cm) | 15 |
| Voltage (kV) | 23.5-29 |
| Temperature, humidity (° C., %) | 24, 21-22 |

### 3. EtOH Gas Visualization Test

(5) The fiber as prepared in accordance with 4) was placed in front of a camera lens, and EtOH-containing gas was applied to the fiber. A variation in the fluorescence of the NADH produced by ADH in response to the EtOH molecules having been trapped by the microfiber mesh was recorded as a video image using a video camera for immediate visualization of the EtOH gas.

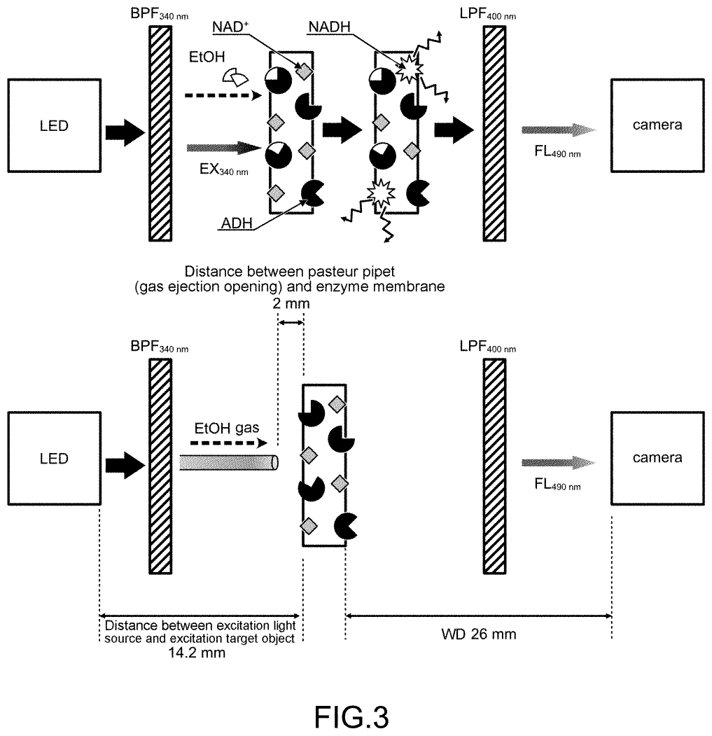

FIG. 3 illustrates a schematic diagram of the experimental system. The distance between an excitation light source and an excitation target object was set as 14.2 mm, and the distance between a gas-applying pasture pipette and an enzyme membrane was set as 2 mm. Three types of gas application conditions as shown in Table 3 were used, where the seconds indicate an elapse of the time in seconds from the start of video recording. The EtOH gas concentration was set as 200 ppm (Preparation conditions: diffusion tube model: D-05; thermostatic chamber temperature: 40° C.; flow rate: 0.6 L/min), and the flowing speeds of EtOH gas and drying gas were set as 100 mL/min. Further, the video was recorded for 180 seconds at 30 fps, where the shutter speed of the camera was set as 33.33 ms, the gain was set as 5.0 and the black level was set as 5.0.

TABLE 3
Gas application conditions

| Conditions: | Used samples | Gas application conditions |
|---|---|---|
| (i) | PVA/ADH/NAD⁺ Blend | EtOH gas had been applied |
| | | from Second 20 to Second 40, |
| | | and air gas had been applied |
| | | after Second 40 |
| (ii) Negative | PVA/ADH/NAD⁺ Blend | Air gas had been applied from |
| control 1 | | Second 20 to Second 40 |
| (iii) Negative | PVA/NAD⁺ Blend | EtOH gas had been applied |
| control 2 | | from Second 20 to Second 40, |
| | | and air gas had been applied |
| | | after Second 40 |

(6) Two Types of Numerical Analysis were Performed for the Obtained Video Image.

One of them is a processing method for calculating a variation in fluorescence intensity over time, and another one of them is a processing method for calculating a rate of the variation in fluorescence intensity thereover; that is, a processing method for calculating a production speed of the NADH. The region of interest (ROI) for numerical analysis was set as 250×250 pixels.

### 4. Test Result

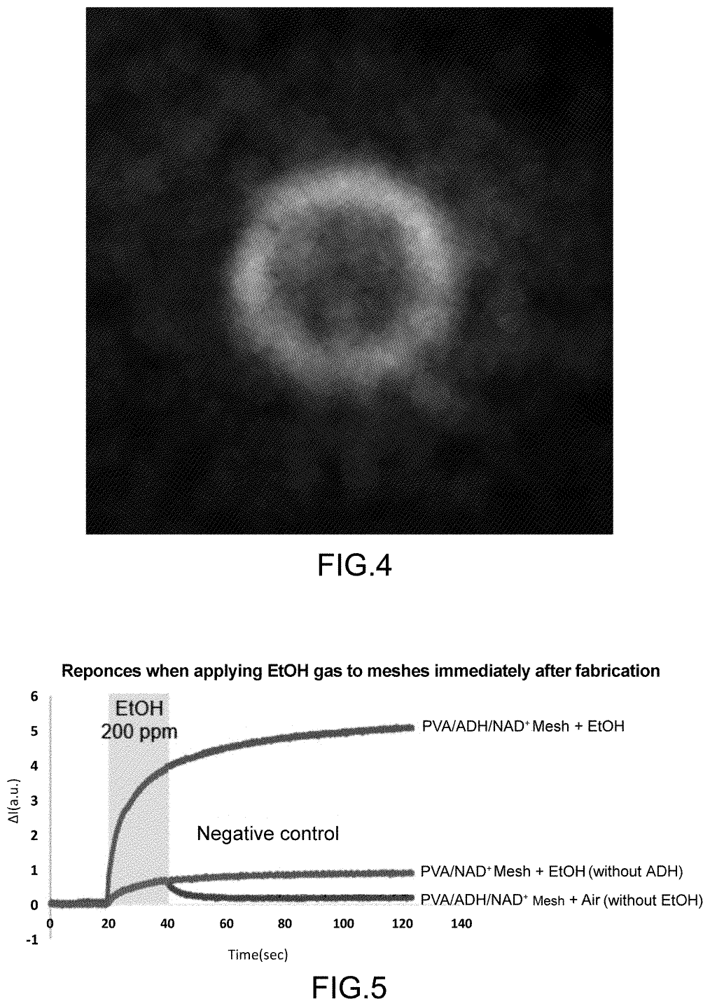

The EtOH gas applied to a microfiber mesh composed of PVA/ADH/NAD⁺ Blend fiber (hereafter referred to as “PVA/ADH/NAD⁺ microfiber mesh”) increased the fluorescence intensity AI (FIGS. 4 and 5), while no increment in the AI was observed when a dry gas was applied thereto (FIG. 5). Further, no AI increment was observed for the PVA/NAD⁺ Blend fiber from which the enzyme had been removed even when EtOH gas as a substrate was applied thereto (FIG. 5). These results therefore show that the increment of AI when applying EtOH gas to the PVA/ADH/NAD⁺ Blend fiber was an event resulting from an enzymatic reaction.

### Working Example 2

### Quantitative Study

A quantitative study of the EtOH contained in a gas phase was conducted using the PVA/ADH/NAD⁺ microfiber mesh which is fabricated by the same method as the working example 1.

### 1. Materials and Devices

The materials and devices same as those of working example 1 were used.

### 2. Fabrication of Microfiber Mesh

(1) 50 mL MilliQ water was put into a beaker and stirred by a stirrer. 2 g of sodium hydroxide was added in small batches to prepare 1 M NaOH.
(2) The 1M NaOH as prepared in (1) was added into a PVA solution to adjust the solution to the pH of 8.
(3) ADH and NAD+ were directly added into the PVA solution as prepared in (2) having a pH of 8 to have the ADH concentration of 3.125 mg/mL and the NAD⁺ concentration of 132.5 mg/mL. The solution concentrations were adjusted such that the amount of NAD⁺ was set as 10 μmol and the amount of ADH was set as 50 units when the spinning amount per one sheet was set as 50 μL. Table 4 shows details.

TABLE 4
Prepared concentrations, spinning amounts
and contents per sheet of ADH and NAD⁺

| | ADH | NAD⁺ |
|---|---|---|
| Concentration (mg/ml) | 3.125 | 132.5 |

| | |
|---|---|
| Flow rate (ml/min) | 0.015 |
| Spinning time (min) | 3.3 |
| Spinning amount (ml) | 0.05 |

| | | |
|---|---|---|
| Content (mg) per sheet | 0.1563 | 6.625 |
| ADH activity (Units/mg) | 321 | — |
| Enzyme activity per sheet (Units) | 50 | — |
| Molecular mass of NAD⁺ (g/mol) | — | 663.43 |
| Molar number of NAD⁺ per sheet | — | 10 |

(4) The solution as prepared in (3) shown above was used to spin a microfiber using electrospinning to thereby fabricate a microfiber mesh. The electrospinning conditions are as shown in Table 5. As a collector, there was used a resin seat having a 1.5 cm by 1.5 cm square hole on which aluminum foil was covered.

TABLE 5
Electrospinning condition

| | |
|---|---|
| Flow rate (ml/min) | 0.015 |
| Spinning time (min) | 3.3 |
| Spinning amount (ml) | 0.05 |
| Spinning height (cm) | 15 |
| Voltage (kV) | 19-21 |
| Temperature, humidity (° C., %) | 23-26, 14-16 |

3. Evaluation of Output Response with Respect to the Variations in Flow Rate (Molar Amount of Applied EtOH) of Applied Gas
(5) The microfiber mesh as fabricated in accordance with (4) was placed in front of a camera lens, and an EtOH-containing gas was applied to the fiber. A variation in the fluorescence of the NADH produced in response to the reaction between ADH and EtOH which is subjected to the molecular trapping in the microfiber mesh was visualized to record the variation by a video camera to thereby measure the EtOH gas. Here, the distance between an excitation light source and an excitation target object was set as 13 mm, and the distance between a gas applying pasteur pipette and an enzyme membrane was set as 2 mm. The EtOH gas concentration was set as 200 ppm (the conditions were: diffusion tube: D-05; Temperature: 40° C.; diluent gas flow rate: 0.6 L/min), and four types of flowing speeds of EtOH gas and drying gas were set as 20, 50, 100 and 200 ml/min (respectively corresponding to the EtOH application amounts of 2.7 nmol/sec, 6.8 nmol/sec, 13.6 nmol/sec and 27.3 nmol/sec), respectively. The EtOH gas was applied for 20 seconds after 20 seconds from the start of the video recording, and then the gas was switched to the dry gas and the video recording was kept until the second 180. Here, the video was recorded at 30 fps, where the shutter speed of the camera was set as 33.33 ms, the gain was set as 5.0 and the black level was set as 5.0.
(6) Two Types of Numerical Analysis were Performed for the Obtained Video Image.

One of them is a processing method for calculating a variation in fluorescence intensity over time, and another one of them is a processing method for calculating a rate of the variation in fluorescence intensity thereover; that is, a processing method for calculating the production speed of the NADH. Here, the region of interest (ROI) for numerical analysis was set as 250×250 pixels.

### 4. Experimental Result

FIG. 6 shows the rate of fluorescence variations; i.e., variations per unit time in differential values of the variation in fluorescence intensity over time in response to the produced NADH when 200 ppm EtOH gases having four types of flow rates 20, 50, 100 and 200 ml/min (respectively corresponding to the EtOH application amounts of 2.7, 6.8, 13.6 and 27.3 nmol/sec) were applied to the PVA/ADH/NAD⁺ microfiber mesh.

FIG. 7 shows a correlation between the rate of fluorescence variation and the amounts of EtOH applied per unit time (2.7 to 27.3 nmol/sec) to the PVA/ADH/NAD⁺ microfiber mesh. The relation between the rate of fluorescence variation and the applied amounts of EtOH per unit time showed favorable linearity, which indicates that the PVA/ADH/NAD⁺ microfiber mesh has excellent quantitativeness for the EtOH content in a gas.

### Working Example 3

### Evaluation of Preservation Stability of PVA/ADH/NAD⁺ Microfiber Mesh

Next, the preservation stability of the PVA/ADH/NAD⁺ microfiber mesh was evaluated.

### 1. Materials and Devices

The materials and devices same as those of the working examples 1 and 2 were used.

### 2. Fabrication of PVA/ADH/NAD⁺ Microfiber Mesh

(1) 50 mL MilliQ water was put into a beaker and stirred by a stirrer. 2 g of sodium hydroxide was added in small batches to prepare 1 M NaOH.
(2) The 1M NaOH as prepared in (1) was added into a PVA solution to adjust the solution to the pH of 8.
(3) ADH and NAD⁺ were directly added into the PVA solution as prepared in (2) having a pH of 8 to have an ADH concentration of 3.125 mg/mL and NAD⁺ concentration of 132.5 mg/mL. The solution concentrations were adjusted such that the amount of NAD⁺ was set as 10 μmol and the amount of ADH was 50 units when the spinning amount per one sheet was set as 50 μL. Table 6 shows details.

TABLE 6
Prepared concentrations, spinning amounts
and contents per sheet of ADH and NAD⁺

| | ADH | NAD⁺ |
|---|---|---|
| Concentration (mg/ml) | 3.125 | 132.5 |

| | |
|---|---|
| Flow rate (ml/min) | 0.015 |
| Spinning time (min) | 3.3 |
| Spinning amount (ml) | 0.05 |

| | | |
|---|---|---|
| Content (mg) per sheet | 0.1563 | 6.625 |
| ADH activity (Units/mg) | 321 | — |
| Enzyme activity per sheet (Units) | 50 | — |
| Molecular mass of NAD⁺ (g/mol) | — | 663.43 |
| Molar number of NAD⁺ per sheet | — | 10 |

(4) The solution as prepared in (3) shown above was used to spin a fiber.

The electrospinning conditions were as shown in Table 7. As a collector, there was used a resin seat having a 1.5 cm by 1.5 cm square hole on which aluminum foil was covered.

TABLE 7
Electrospinning condition

| | |
|---|---|
| Flow rate (ml/min) | 0.015 |
| Spinning time (min) | 3.3 |
| Spinning amount (ml) | 0.05 |
| Spinning height (cm) | 15 |
| Voltage (kV) | 19-21 |
| Temperature, humidity (° C., %) | 23-26, 12-16 |

(5) Morphological observation by Scanning Electron Microscope (SEM)

Morphologies of the PVA/ADH/NAD⁺ microfiber meshes were observed by a scanning electron microscope (SEM) on the day when the PVA/ADH/NAD⁺ Blend fiber was spun, and the day after the PVA/ADH/NAD⁺ Blend fiber had been preserved at 4° C. for 14 days.
(6) Preservation evaluation of enzyme activity of enzyme membrane

As controls, visualization of EtOH gas (as a positive control) and application of dry gas (as a negative control) were performed on the fibers immediately after fabrication. The distance between an excitation light source and an excitation target object was set as 13 mm, and the distance between a gas-applying pasteur pipette and an enzyme membrane was set as 2 mm. The EtOH gas concentration was set as 200 ppm (the conditions were: diffusion tube: D-05; Temperature: 40° C.; diluent gas flow rate: 0.6 L/min), and the flowing speeds of EtOH gas and drying gas were set as 100 mL/min. The EtOH gas was applied for 20 seconds after 20 seconds from the start of video recording, and then the gas was switched to a dry gas and the video recording was kept until Second 180. Here, the video was recorded for 180 seconds at 30 fps, where the shutter speed of the camera was set as 33.33 ms, the gain was set as 5.0, and the black level was set as 5.0. The samples to be measured after two weeks were put into φ35 mm dishes, which were further put into a zipped plastic bag and sealed to be stored at 4° C. in a refrigerator. After two weeks, the EtOH gas was subjected to visualization with the same steps using fibers, immediately after fabrication (as a control), and the stored fibers.
(7) Two types of numerical analysis were performed for the obtained video image.

One of them is a processing method for calculating a variation in fluorescence intensity over time, and another one of them is a processing method for calculating a rate of the variation in fluorescence intensity thereover; that is, a processing method for calculating the production speed of the NADH. Here, the region of interest (ROI) for numerical analysis was set as 250×250 pixels.

### 3. Experimental Result

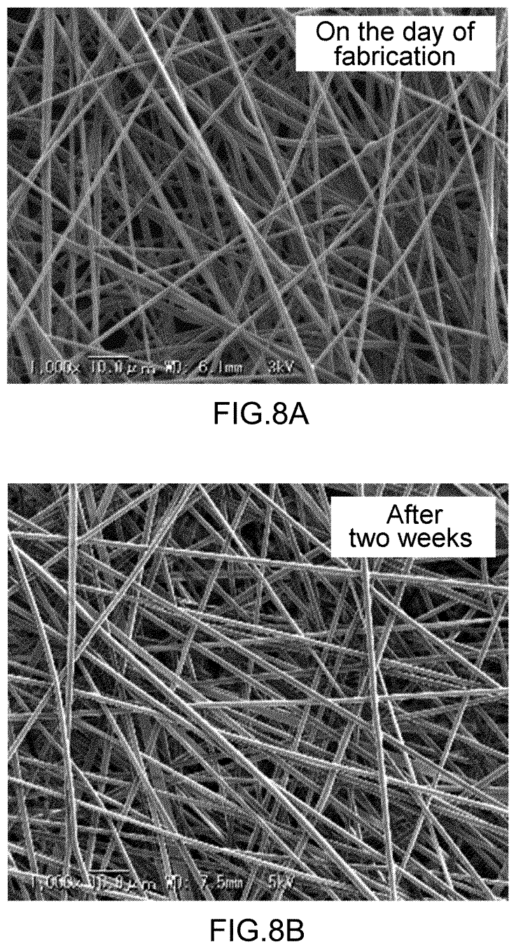

No change was observed in the electron scanning microscope images between the PVA/ADH/NAD⁺ Blend fiber mesh on the day of fabrication (FIG. 8A) and that of 14 days after the fabrication (FIG. 8B).

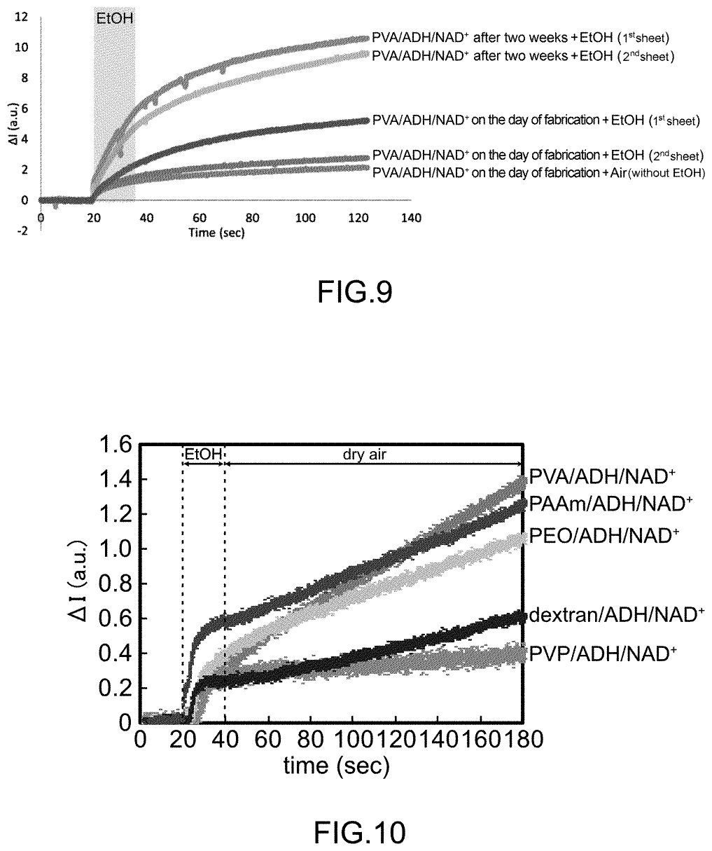

FIG. 9 shows the variations in fluorescence intensities when EtOH gas was visualized using the PVA/ADH/NAD⁺ Blend fiber mesh immediately after the fabrication and the PVA/ADH/NAD⁺ Blend fiber mesh having stored at 4° C. in a refrigerator for two weeks after the fabrication. The PVA/ADH/NAD⁺ Blend fiber meshes showed variations in fluorescence intensity which were based on the EtOH trapping effects that are favorable after having been preserved at 4° C. for 14 days after the fabrication.

This characteristic will be considered as a great advantage in the industrial application of the molecular trapping material according to the present invention because such characteristic has not been observed in a conventional method (Patent document 1) which utilizes a cotton mesh for immobilizing the ADH (in the case of cotton mesh, an enzyme deactivates and the visualization is therefore made impossible on the next day even when it is preserved at 4° C. in a refrigerator).

### Working Example 4

### Evaluation and Fabrication of Microfiber Mesh Incorporating ADH and NAD⁺ in Various Types of Water-Soluble Polymer.

Microfiber meshes into which ADH and NAD⁺ were mixed were fabricated for not only a water-soluble polymer of PVA but also for other types of water-soluble polymers to study the measurement by visualization of EtOH gas to evaluate utilization of the various types of water-soluble polymers for the gasborne substance trapping material.

### 1. Materials and Devices

The materials and devices same as those of the working examples 1 to 3 were used except for the materials and devices as listed in the following:
PVP: Polyvinylpyrrolidone K-90, Mv: 360,000, NACALAI TESQUE, INC., CAS No. 9003-39-8
PEO: Poly(ethylene oxide), Mv: ˜900,000, Sigma-Aldrich, CAS No. 25322-68-3
Dextran: Dextran 70, Mw: ca. 70,000, Cas No. 9004-54-0
PAAm: Polyacrylamide, Mn: 150,000, ALDRICH (PAAm (Low molecular weight))

### 2. Experimental Method

### (1) Preparation of Water-Soluble Polymer Solutions

Each of the aqueous solutions of PVP, PEO, Dextran and PAAm (Low molecular weight) underwent concentration adjustment to have a viscosity that is comparable to that of the undiluted solution of PVA solution of Arabic Yamato (Registered trademark) liquid glue.

### (i) Preparation of PVA Aqueous Solution

PVA solution of Arabic Yamato (Registered trademark) liquid glue was used in the same manner as the working examples 1 to 3.

### (ii) Preparation of PVP Aqueous Solution

PVP was put in a 20 mL vial to which MilliQ water was added, and the mixture was stirred for more than overnight under the light shielding condition to prepare a 25 w/w % PVP aqueous solution.

(iii) Preparation of PEO Aqueous Solution

PEO was put in a 20 mL vial to which MilliQ water was added, and the mixture was stirred for more than overnight under the light shielding condition to prepare a 6 w/w % PEO aqueous solution.

### (iv) Preparation of Dextran Aqueous Solution

Dextran was put in a 20 mL vial to which MilliQ water was added, and the mixture was stirred for more than overnight under the light shielding condition to prepare a 50 w/w % dextran aqueous solution.

### (v) Preparation of PAAm (Low Molecular Weight) Aqueous Solution

PAAm (Low molecular weight) was put in a 20 mL vial to which MilliQ water was added, and the mixture was stirred for more than overnight under the light shielding condition to prepare a 15 w/w % PAAm (Low molecular weight) aqueous solution.

Next, 0.1N NaOH aqueous solution was added to each of the aqueous solutions of PVA, PVP, PEO, Dextran and PAAm (Low molecular weight) to control the pH close to 8.

### (2) Preparation of Raw Material Aqueous Solutions for Respective Microfiber Meshes

Each of the 50 μL/mesh (preparation amount: 0.8 mL) water-soluble polymer aqueous solutions (pH 8) as outlined above was mixed with 50 units/mesh (preparation amount: 2.56 mg) of ADH and 10 μL/mesh (preparation amount: 106 mg) of NAD⁺ to prepare a raw material aqueous solution for each of the respective microfiber meshes.

### (3) Microfiber Mesh Fabrication by Electrospinning

The raw material aqueous solutions prepared in (2) for the respective microfiber meshes were used to perform electrospinning under the conditions of Table 7 to fabricate the microfiber meshes of the respective water-soluble polymers.

TABLE 8
Electrospinning condition

| | | | | | PAAm |
|---|---|---|---|---|---|
| | | | | | (Low molecular |
| | PVA | PVP | PEO | Dextran | weight) |
| Flow rate (ml/hr) | 0.9 | | | | |
| Spinning time (min) | 3.33 | | | | |
| Spinning amount (ml) | 0.05 | | | | |
| Spinning height (cm) | 15 | | | | |
| Voltage (kV) | 21-23 | 25-27 | 16-20 | 20-25 | 20 |
| Temperature/humidity | 21/36 | 25/49 | 22/16 | 22/22 | 22/14 |
| (° C./%) | | | | | |

### (4) Visualization Experiment by EtOH Gas Application

200 ppm of EtOH gas was applied for 20 seconds to each microfiber mesh that was fabricated by mixing ADH, NAD⁺ and one of the respective water-soluble polymers having been fabricated as above, and the intensity of 490 nm fluorescence having been emitted upon irradiation of 340 nm excitation light was measured to evaluate if the ADH remains active not only in the PVA water-soluble polymer but also in other types of water-soluble polymers.

Specifically, the fiber as fabricated in accordance with (3) was placed in front of a camera lens, and an EtOH gas was applied to the fiber to promptly visualize and measure the EtOH gas. The EtOH gas concentration was set as 200 ppm, and the flowing speeds of EtOH gas and drying gas were set as 100 ml/min. The video was recorded for 180 minutes at 30 fps, where the shutter speed of the camera was set as 33.33 ms, the gain was set as 5.0 and the black level was set as 5.0.

### 3. Experimental Results

FIG. 10 shows variations in intensity of 490 nm fluorescence emission light emitted from the microfiber mesh in response to the irradiation of 340 nm excitation light when EtOH gas was applied to each of the microfiber meshes of the respective water-soluble polymers.

As shown in FIG. 10, the microfibers of PEO and PAAm (low molecular weight) exhibited variations in fluorescence intensity which were comparable to that of microfiber mesh of PVA. Meanwhile, although PVP and dextran exhibited small variations in fluorescence intensity, they showed increments in fluorescence intensity.

These results indicate that the ADH incorporated therein remains active when it is incorporated not only in the PVA but also in other types of water-soluble polymer materials.

### INDUSTRIAL APPLICABILITY

The gasborne substance trapping material according to the present invention can measure substances such as VOCs contained in a gas phase, and therefore may be used for a variety of gas sensors.

Examples of such gas sensor include a biosensor. For example, there may be used a biosensor for measuring ethanol (EtOH) for alcoholism treatment, a biosensor for measuring acetaldehyde for risk assessment of oral or esophageal cancer, a biosensor for measuring methanol for evaluation of intestinal environment, a biosensor for measuring acetone for evaluation of diabetes and lipid metabolism, a biosensor for measuring isopropanol for evaluation of diabetes and lipid metabolism, a biosensor for measuring formaldehyde for diagnosis of lung cancer, a biosensor for measuring ammonia for diagnosis of liver diseases and a biosensor for measuring dimethyl sulfide for evaluating halitosis or oral environment.

The gasborne substance trapping material according to the present invention may be used not only as a gas sensor but also for purifying the air by trapping a substance floating in the air. Further, the gasborne substance trapping material according to the present invention may be used for recovering a desired substance by selectively trapping the substance floating in the air. Further, the trapping/recovering of the substance floating in the air allows measurement of the cleaning or contamination level of a gas in the air. Furthermore, the trapping of a pathogen substance floating in the air may prevent affection with a disease.

1. A gasborne substance trapping material for trapping a substance contained in a gas phase, comprising a water-soluble polymer having a functional protein that retains a capability to bind to the substance, wherein the functional protein exhibits a capability to bind to the substance to trap the substance.

2. The gasborne substance trapping material according to claim 1, wherein the functional protein retains a function in the water-soluble polymer.

3. The gasborne substance trapping material according to claim 1 or 2, wherein the functional protein is an enzyme or an antibody.

4. The gasborne substance trapping material according to any one of the preceding claims, wherein the substance is a volatile organic compound (VOC).

5. The gasborne substance trapping material according to any one of the preceding claims, wherein the functional protein is an enzyme, and the water-soluble polymer is further mixed with a cofactor.

6. The gasborne substance trapping material according to any one of the preceding claims, wherein the water-soluble polymer is selected from polyvinyl alcohol (PVA), polyethylene oxide (PEO), polyacrylamide (PAAm), polyvinylpyrrolidone (PVP), dextran and polyethylene glycol (PEG).

7. The gasborne substance trapping material according to any one of the preceding claims, wherein the water-soluble polymer is in the form of a microfiber or a thin film.

8. The gasborne substance trapping material according to any one of claims 5 to 7, wherein the volatile organic compound is ethanol, the enzyme is an alcohol dehydrogenase and the cofactor is NAD⁺ (Oxidized Nicotinamide adenine dinucleotide).

9. A biosensor for trapping and measuring a substance contained in a gas phase, wherein the biosensor employs the gasborne substance trapping material according to any one of the preceding claims.

10. A gasborne substance trapping method for trapping a substance contained in a gas phase with a water-soluble polymer having a functional protein, wherein the functional protein exhibits a capability to bind to the substance to trap the substance.
